(19) United States
(12) Patent Application Publication
(10) Pub. No.: US 2010/0278940 A1
(43) Pub. Date: Nov. 4, 2010

(54) Calcium supplements

(75) Inventors: Dean Neuls, Vancouver (CA); Marcos Antonio Dias Neves, Praia Do Canto (BR)

Correspondence Address: NIXON & VANDERHYE, PC, 901 NORTH GLEBE ROAD, 11TH FLOOR, ARLINGTON, VA, 22203, US

(73) Assignees: HL DISTRIBUTION COMPANY, Las Vegas, NV (US); ALGAECAL DISTRIBUTION INC.

(21) Appl. No.: 12/662,662

(22) Filed: Apr. 27, 2010

Related U.S. Application Data
(63) Continuation-in-part of application No. 12/289,014, filed on Oct. 17, 2008.
Application No. 12/289,014 is a division of application No. 11/298,165, filed on Dec. 8, 2005, now abandoned.
(60) Provisional application No. 60/668,438, filed on Apr. 4, 2005.

(51) Int. Cl. A61K 33/30; A61K 33/24; A61P 19/00; A61P 19/10; A61P 19/02

(52) U.S. Cl. 424/643; 424/646

(57) ABSTRACT

A composition comprising at least 15%, by weight, of calcium derived from a rhodolith coralline algae; magnesium; strontium; vanadium; vitamin K; and vitamin D3.

### FIELD OF THE INVENTION

This invention relates to calcium compositions. In one preferred aspect, the invention relates to dietary supplements that comprise calcium.

### BACKGROUND OF THE INVENTION

Calcium is an essential mineral and is one of the most popular dietary supplements in the United States. Almost all calcium in the human body is found in the bones and teeth where it is critical for health and strength. Calcium is also found in the blood, extracellular fluid, muscles, and other tissues, where it plays a role in blood pressure, muscle contraction, transmission of nerve impulses, and glandular secretions. Evidence from recent studies suggests that calcium, besides maintaining bone strength, may also be good for the heart, help lower blood pressure, and improve blood lipid levels. An American Cancer Society study also found that calcium may reduce the risk of colorectal cancer.

Experts recommend a daily dose of calcium of 1,000 milligrams for men and premenopausal women (post-menopausal women need 1,200 to 1,500 milligrams). Such a dose is obtainable from dietary sources such as milk, dairy products, and leafy green vegetables but it has been found that the typical American woman gets 625 milligrams and the typical man 865. Therefore, there exists a need for dietary supplements that contain calcium.

Many types of calcium supplements are available in a variety of forms, including tablets, caplets, syrups, chewable tablets, softgels, soft chewable cubes, and antacids. There are also calcium fortified foods and juices. The most common form of calcium used in dietary supplements is calcium carbonate, although there are many different types of calcium available, including calcium malate, calcium citrate malate, calcium lactate, and calcium gluconate. These sources of calcium show varying degrees of bioavailability which may limit their potency. In addition, certain forms of calcium can cause constipation.

In addition to calcium there are many other minerals and other materials that are desirable in a supplement.

One mineral that is useful in a supplement is magnesium. Magnesium is utilized in a variety of essential bodily functions such as ATP metabolism, muscle contraction, nerve function, transmembrane transportation, and enzyme function. Another useful material for a supplement is strontium. It has been suggested that strontium can aid healthy bones.

Many consumers of supplements prefer that the products be derived from ‘natural’ sources. Marine sources are particularly attractive to consumers due to the perceived health benefits. Therefore, it would be advantageous to provide a supplement derived from a marine source that is easily harvested and processed.

### SUMMARY OF THE INVENTION

The present invention relates to calcium compositions. In one preferred aspect the present invention relates to dietary supplement compositions derived from algae, preferably marine algae, that comprise calcium. The present invention also relates to methods of treatment using such compositions. The present invention also relates to methods of processing such compositions. The present invention also relates to kits comprising such compositions.

As used herein, “algae” refers to a non-flowering, stemless, water plant. The term “marine algae” refers to those algae that spend a significant portion of their life cycle in a marine environment.

The present invention is useful for the treatment of a variety of conditions. As used herein, “treatment” means any manner in which the symptoms of a condition are ameliorated or otherwise beneficially altered. Treatment also encompasses prophylaxis. For example, the present invention can be useful for preventing relapse in patients who have previously been cured of the condition.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more.”

### DETAILED DESCRIPTION OF THE INVENTION

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The compositions of the present invention comprise at least 5%, by weight, calcium. Preferably, the present compositions comprise at least 10%, more preferably at least 15%, even more preferably at least 20%, even more preferably still at least 25%, by weight, calcium.

Preferably the compositions herein comprise less than 65%, by weight, more preferably less than 45%, by weight, even more preferably less than 35%, by weight, calcium.

Preferably, at least a portion of the calcium in the present compositions is chelated.

The present compositions preferably comprise magnesium. Preferably, the present compositions comprise at least 1%, more preferably at least 3%, even more preferably at least 5%, even more preferably at least 6%, even more preferably at least 7%, even more preferably at least 8%, by weight, magnesium.

Preferably the compositions herein have weight ratio of calcium to magnesium of less than 5:1, more preferably less than 4:1, and even more preferably less than 3:1.

The present composition preferably comprise one or more vitamins. Preferred vitamins include vitamin C, vitamin D, vitamin K, and combinations thereof.

The present compositions preferably comprise carbon. Preferably, the present compositions comprise at least 2%, more preferably at least 5%, even more preferably at least 10%, by weight, carbon.

The present compositions are derived from marine algae. Any suitable marine algae may be used. Preferred are marine algae of the division Rhodophyta (red algae). Preferred are marine algae of the class Rhodophyceae. Preferred are marine algae of the order Corallinale. Preferred are non-geniculate Coralline algae. Preferred are rhodoliths.

Preferably the present compositions comprise other minerals including, but not limited to, chromium, copper, iron, manganese, potassium, strontium, sulfur, zinc, and combinations thereof. It is preferred that the compositions herein comprise strontium. One preferred form of strontium for use herein is strontium citrate. Preferably, the compositions herein comprise at least 25 minerals, more preferably at least 50 minerals, even more preferably at least 70 minerals.

The present composition may comprise other nutritional ingredients. For example, phytonutrients, fatty acids such as such as omega 3, 6, and 9, digestive enzymes such as Betaine HCL, Casein Phospho Peptide (CCP), and combinations thereof.

The compositions herein can be presented in any suitable form including, but not limited to, powders, capsules, tablets, wafers, pastes, gels, liquids, or suspensions.

The present compositions can be prepared in any suitable way. Preferably the compositions of the present invention are manufactured by harvesting the algae, grinding, and sterilizing. It is preferred that the present compositions do not require any reduction of sodium content prior to use. The powder can be encapsulated with a suitable capsule, and packing the capsules in a form suitable for sale to consumers. Preferably the capsules comprise at least 400 mg, more preferably at least 550 mg, of composition.

The compositions herein may be presented in the form of a ‘functional food’. For example, the present compositions can be added to cookies, bread, sports drinks, meal replacement drinks, other soft drinks, and the like. These product presentations can be useful to help subjects who are reluctant to taking a capsule or tablet to ingest the product.

The compositions of the present invention can be used as a dietary supplement. The present compositions are preferably taken on a daily basis. Preferably, at least 1000 mg, more preferably at least 1500 mg, even more preferably at least 2000 mg, of composition is taken daily.

The present invention also relates to kits comprising the composition. The present kit preferably comprises composition as described herein and instructions for ingesting said composition. Preferably the instructions recommend ingestion on a regular, preferably daily, basis. The present kits can comprise other compositions. If present the other compositions preferably comprise strontium, vitamin D, or combinations thereof. Preferably the present kits comprise a composition according to Example 1 and a composition comprising strontium citrate.

The compositions of the present invention can be used to treat a variety of disorders and for a variety of purposes. In particular, the present compositions can be useful in treating osteoporosis, arthritis, bone spurs, plantar fasciitis, or other musculoskeletal disorders, to aid athletic performance, aid recover from injury, improve strength or aid overall physical condition.

The present compositions can be used to aid wound healing or to improve the condition of skin. For example, the present compositions can be used to treat ulcers so as diabetic ulcers or bed sores. Preferably the compositions are applied topically for such uses. For instance, the compositions can be applied to a wound such as an ulcer in the form of a powder, a gel, a paste, or other suitable form. Bandages may also be treated with the present compositions and then applied to the affected area. The present compositions can also be used to treat burns.

The compositions herein are useful for cosmetic applications. They may be used alone or in combination with other ingredients to improve the condition or appearance of the skin.

The compositions of the present invention are useful for treating non-human animals. For example, companion animals such as dogs and cats, working animals such as racehorses and greyhounds, and farm animals such as cattle and sheep may be treated with the present compositions. The present compositions are of particular benefit in helping companion animals suffering from the effects of age or genetic disorders and working animals in improving or maintaining performance.

The present compositions are useful for a variety of other purposes. For example, the present compositions may be used as a pH buffer, in laundry or homecare products, in water filtration, for treating oil or chemical spills, or for improving smell or taste.

### EXAMPLES

It will be understood that the following embodiments of the present invention are intended to be illustrative of some of the possible applications or principles. Various modifications may be made by the skilled person without departing from the true spirit and scope of the invention.

### Example 1

A composition is produced from non-geniculate Coralline algae by harvesting the algae, grinding to a powder, adding 200 IU of vitamin D3 and 1.25 mg of vitamin K-2 (menaquinone), encapsulating 600 mg the powder in a Vcap™ capsule, and packing 90 capsules in a bottle, said composition having the following mineral content:

| | PPM |
|---|---|
| Aluminum | 1420 |
| Antimony | 4.57 |
| Arsenic | 0.11 |
| Barium | 5.57 |
| Beryllium | 0.081 |
| Bismuth | 2.19 |
| Boron | 12.1 |
| Bromine | 11.5 |
| Cadmium | <0.03 |
| Calcium | 279,000 |
| Carbon | 124,000 |
| Cerium | 2.11 |
| Cesium | 2.66 |
| Chloride | 2,370 |
| Chromium | 5.1 |
| Cobalt | 0.107 |
| Copper | 1.59 |
| Dysprosium | 1.15 |
| Erbium | 18.9 |
| Europium | <0.1 |
| Fluoride | 7.55 |
| Gadolinium | 3.04 |
| Gallium | 1.27 |
| Germanium | 3.08 |
| Gold | <0.05 |
| Hafnium | <0.1 |
| Holmium | <0.1 |
| Indium | 0.17 |
| Iodine | 10.6 |
| Iridium | <0.05 |
| Iron | 10,100 |
| Lanthanum | 0.314 |
| Lead | 0.061 |
| Lithium | 3.9 |
| Lutetium | 0.31 |
| Magnesium | 99,800 |
| Manganese | 90.5 |
| Mercury | 0.011 |
| Molybdenum | <0.1 |
| Neodymium | 3.51 |
| Nickel | 0.074 |
| Niobium | <0.1 |
| Osmium | <0.05 |
| Palladium | <0.05 |
| Phosphorus | 169 |
| Platinum | <0.05 |
| Potassium | 427 |
| Praseodymium | 0.63 |
| Rhenium | <0.2 |
| Rhodium | <0.05 |
| Rubidium | 27.6 |
| Ruthernium | 0.055 |
| Samarium | 0.67 |
| Scandium | 0.685 |
| Selenium | 0.06 |
| Silica | 28,000.00 |
| Silver | 1.78 |
| Sodium | 3970 |
| Strontium | 2190 |
| Sulfur | 940 |
| Tantalum | <0.05 |
| Tellurium | 0.066 |
| Terbium | <0.2 |
| Thallium | 0.33 |
| Thorium | 0.081 |
| Thulium | <0.05 |
| Tin | 0.197 |
| Titanium | 31.1 |
| Tungsten | <0.05 |
| Vandaium | 12.9 |
| Ytterbium | 0.237 |
| Yrrtirum | 1.74 |
| Zinc | 16.2 |
| Zirconium | 0.642 |

### Example 2

A composition is produced from non-geniculate Coralline algae by harvesting the algae and grinding to a powder. The composition comprises:

| | Wt. % |
|---|---|
| Calcium | 31.7 |
| Magnesium | 8.3 |
| Carbon | 11.4 |
| Iron | 1.6 |
| Other material | 47.0 |

### Example 3

A kit comprising 90 capsules of a composition according to Example 1 and instructions for ingesting four capsules daily.

### Example 4

A kit comprising 120 capsules of a composition according to Example 1 and 90 capsules comprising 743 mg strontium citrate with instructions for ingesting four capsules daily of the former and three capsules daily of the latter.

### Example 5

A 64 year old female osteoporosis patient loses an average of 1% of her bone mineral density per year. She takes a daily dose of 4 capsules according to Example 1. In addition, she takes 2230 mg per day of strontium citrate. After 10 weeks of following this regimen her physician measures her bone mineral density at her hip by dual X-ray absorptometry and finds it is increased by 5%. Her physician tells her that the bone weakness caused by her osteoporosis is reversing.

### Example 6

A 57 year old male is suffering from acid reflux disease. The patient takes a daily dose of 4 capsules according to Example 1. After 3 months the man reports that he no longer experiences acid reflux even after eating his favorite pepperoni pizza.

### Example 7

A 32 year old male athlete is training for a triathlon. Due to overtraining the athlete has recurrent pain in his left hip and lower back. The athlete takes a daily dose of 2400 mg of a composition according to Example 1 for 4 weeks. The pain in his lower back is completely cured while the pain in his left hip is significantly reduced.

### Example 8

A diabetic 62 year old women has a persistent ulcer on her right leg. She applies 2 g twice a day of a composition according to Example 2 and, after two weeks, finds that the ulcer begins to heal.

### Example 9

A 10 year old male Alsatian dog is diagnosed with osteoarthritis in his left rear hip. 1200 mg of a composition according to Example 2 is given to the dog along with his food. After three weeks the dog shows a marked improvement in his mobility and general condition.

### Example 10

A 5 year old thoroughbred race horse is consistently posting good times on race day. 10 grams daily of a composition according to Example 1 is given to the horse along with his feed. At the following race the horse posts a personal best time.

### Example 6

A 57 year old male is suffering from acid reflux disease. The patient takes a daily dose of 4 capsules according to Example 1. After 3 months the man reports that he no longer experiences acid reflux even after eating his favorite pepperoni pizza.

### Example 7

A 32 year old male athlete is training for a triathlon. Due to overtraining the athlete has recurrent pain in his left hip and lower back. The athlete takes a daily dose of 2400 mg of a composition according to Example 1 for 4 weeks. The pain in his lower back is completely cured while the pain in his left hip is significantly reduced.

### Example 8

A diabetic 62 year old women has a persistent ulcer on her right leg. She applies 2 g twice a day of a composition according to Example 2 and, after two weeks, finds that the ulcer begins to heal.

### Example 9

A 10 year old male Alsatian dog is diagnosed with osteoarthritis in his left rear hip. 1200 mg of a composition according to Example 2 is given to the dog along with his food. After three weeks the dog shows a marked improvement in his mobility and general condition.

### Example 10

A 5 year old thoroughbred race horse is consistently posting good times on race day. 10 grams daily of a composition according to Example 1 is given to the horse along with his feed. At the following race the horse posts a personal best time.

### Example 11

An algae-sourced form of calcium derived from a rhodolith coralline algae (AC, also known as DNO361) was obtained from AlgaeCal®, Vancouver, Canada. Dulbecco's Modified minimal Essential Medium (DMEM) and fetal bovine serum (FBS) were purchased from Gibco BRL (Grand Island, N.Y.). Calcium carbonate, calcium citrate and vitamin D₃ were procured from Sigma Chemicals (St. Louis, Mo., USA). All other reagents were of the highest commercial grade available and purchased from Sigma Chemicals (St. Louis, Mo., USA).

### Cell Line and Procedures

Human fetal osteoblast cells (hFOB 1.19-ATCC number: CRL-11372) were seeded at a density of 10,000 cells cm⁻² in a mixture of Dulbecco's modified eagle medium (DMEM) and Ham F12 medium (1:1 ratio) supplemented with 10% fetal bovine serum (FBS) and 0.3 mg/ml G408. Osteoblast cells were cultured in a humidified atmosphere of 5% CO2 at 33.5° C. for cell attachment and proliferation and at 39.5° C. for other assays. These alterations in temperature were required because hFOB 1.19 cells were conditionally immortalized with a gene encoding a temperature-sensitive mutant (tsA58) of SV40 large T antigen.

### AC, Calcium Carbonate, Calcium Citrate and Vitamin D₃ Preparation

The content of known bone supporting minerals in AC is given in the table below:

| | AC | | |
|---|---|---|---|
| | Average | | Amount in |
| Mineral | PPM | Average % | 2400 mg AC |
| Boron | 60.2 | 0.0066 | 0.1584 mg |
| Calcium | 302,200.0 | 30.220 | 725.28 mg |
| Copper | 154.997 | 0.0155 | 0.372 mg |
| Phosphorous | 1021.75 | 0.1022 | 2.453 mg |
| Potassium | 891.25 | 0.0891 | 2.138 mg |
| Magnesium | 45,040.0 | 4.504 | 108.10 mg |
| Manganese | 86.35 | 0.0086 | 0.0206 mg |
| Nickel | 0.224 | 0.000022 | 0.528 mg |
| Selenium | 0.95 | 0.000095 | 0.0023 mg |
| Silica/Silicon | 3,509.25 | 0.3509 | 8.422 mg |
| Strontium | 2482.5 | 0.2483 | 5.959 mg |
| Vanadium | 79.25 | 0.0079 | 189.6 mg |
| Zinc | 5.12 | 0.0005 | 0.012 mg |

AC was dissolved in HCl:H₂O mixture at the ratio of 13:2. Calcium carbonate and calcium citrate were dissolved in distilled water. The stock solution of vitamin D₃ was dissolved in ethyl alcohol as per the manufacturer's instructions and directly added in the medium to achieve its final concentration. The optimal dose of AC was found to be 0.5 mg/ml. The same dose was used for further experiments in the present study. The amount of calcium present in 0.5 mg of AC is 0.15 mg. Hence, this particular dose of 0.15 mg/ml of calcium from calcium carbonate and calcium citrate was used as the effective dose in further experiments. Finally, the pH was maintained same in all the preparations.

### Dose Determination of Calcium Supplements by MTT Assay

Cultured osteoblast cells, at 90% confluence, were treated with different concentrations (0.1, 0.25, 0.5 and 1 mg/ml) of AC for 4 days. After 4 days, cells were washed with warm RPMI-1640 without phenol red, and then MTT (0.5 mg/ml) reagent was added into the wells and incubated for 1 h at 37° C. Then 100 μA of DMSO was added to each well and mixed well. Absorbance of the converted dye was measured at OD of 570 nm.

### Alkaline Phosphatase (ALP) Activity

ALP activity was determined by enzymatic assay. Osteoblast cells, at 90% confluence, were treated with 0.5 mg/ml of AC, 0.15 mg/ml of calcium from calcium carbonate and calcium citrate for 4 days. After treatment, cells were rinsed with PBS, then lysed into 0.6 ml of buffer containing 10 mM Tris-HCL pH 7.5, 0.5 mM MgCl2 and 0.1% Triton X-100. Cell lysate was centrifuged at 2,000×g and the soluble fraction was used for enzyme assay. 50 μL of sample volumes were added to 125 μL glycine buffer (25 mM, pH 9.4), containing 2 mM MgCl2 and 5 mM p-nitrophenylphosphate (pNPP), and incubated at 37° C. for 50 min in a water bath. The enzymatic reaction was stopped by addition of 125 μL 1 M NaOH. The final product (p-nitrophenol) was quantified at 405 nm in a spectrophotometer. The results were normalized by the amount of cells and by specific activity (nmol p-nitrophenol/min/mg/of protein). Total protein content was determined by the BCA method in aliquots of the same samples and calculated in comparison with series of bovine albumin serum as internal standards. Cultures from four independent experiments were analyzed.

### DNA Synthesis Assay

The cultured osteoblast cells, at 90% confluence, were treated with AC, calcium carbonate and calcium citrate with and without vitamin D3 for 4 days. After treatment, the cells were washed with PBS, and then added 1 μCi/ml of [³H] thymidine in the serum free medium into the cell for 4 h at 37° C. Then the cells were rinsed with 2×PBS, and then extracted twice with 10% TCA and lysed in 0.5 N NaOH. Liquid scintillation counting was performed to measure radioactivity in the lysates by using Beckman LS 6500 Multipurpose Scintillation counter, USA.

### Immunocytochemistry for Proliferating Cell Nuclear Antigen (PCNA) Expression

To evaluate proliferative activity, immunohistochemistry for PCNA was performed after 4 days treatment of AC, calcium carbonate and calcium citrate. The experiment was done by following the instructions of PCNA staining kit (Invitrogen). Briefly, the osteoblast cells, at 90% confluence in immunocyto chambers, were incubated for two hours at room temperature with anti-PCNA monoclonal antibody and the labeled polymer method was applied following the manufacturer's protocol. Immunoreactivity was visualized with diaminobenzidine hydrochloride, followed by counterstaining with Meyer's hematoxylin.

### Mineralization/Calcium Deposition Assay

The osteoblast cells were treated, at 90% confluence, with culture medium containing AC, calcium carbonate and calcium citrate with and without vitamin D3. After 2 days treatment, the cells were washed with PBS and they were fixed with 70% ethanol for 1 h, and then stained with 40 mM Alizarin Red S for 10 min and shaken gently. To quantify the bound dye, the stain was solubilized with 10% cetyl pyridinium chloride by shaking for 15 min. The absorbance of the solubilized stain was measured at 561 nm.

### MDA Assay

After 24 h of treatment with compounds in the presence and absence of H₂O₂ (0.3 mM), the cells were washed with cold PBS, scraped, and homogenized in lysis buffer. 200 μA of cell lysate was used for measuring the MDA levels by following the manufacturer's instructions. In brief, the binding of thiobarbituric acid to malondialdehyde-bis-(dimethylacetal) 1,1,3,3-tetramethoxy-propan (MDA) formed during lipid peroxidation results in a chromogenic complex, which was measured at 586 nm by using spectrophotometer. The BIOXYTECH MDA-586 kit (OXISResearch™, CA, USA) was used to determine lipid peroxidation, which increases as a result of oxidative stress. In this study, MDA standard was used to construct a standard curve.

### Statistical Analysis

Data were expressed as the mean±SEM of 3-6 independent experiments. Statistical comparisons of the results were made using one way analysis of variance (ANOVA). Significant differences (p≦0.05) between the means of control and test group were analyzed by the Neuman-Keuls multiple comparison test.

### Optimum Dose Fixation of AC by MTT Assay

In order to establish the optimum dosage, we treated the cells for four days at various concentrations of 0.1, 0.25, 0.5 and 1 mg/ml of AC. The most effective dose of AC was found to be 0.5 mg/ml. We found that 0.5 mg/ml showed significantly better cell survivability by MTT assay at different time points including 4 days treatment. Since 4 days is the maximum time point we have considered for other experiments related to the study we have provided only 4 days data. The same dose was used for further experiments in the present study. The amount of calcium (the most essential mineral for bone growth) present in 0.5 mg of AC is 0.15 mg. Hence, this particular dose of 0.15 mg/ml of calcium from calcium carbonate and calcium citrate was used as the effective dose in further experiments.

Effect of AC on ALP Activity with the Comparison of Calcium Carbonate and Calcium Citrate

Alkaline phosphatase is an enzyme attached to the cell membrane of the osteoblasts where it increases inorganic phosphate concentration in the mineralization of extracellular vessels, favoring the precipitation of calcium phosphate, the main component of the mineral phase of bones. It is also known to be involved in bone mineralization. The activity of ALP, examined after four days treatment, was found to significantly increase in AC treated osteoblasts when compared to control (761±59.4 vs 189.5±6.1 nmol/mg protein, p≦0.05) cells, respectively. Moreover, the effect of AC on ALP activity was significantly higher when compared with calcium carbonate (761±59.4 vs 372.2±15.7 nmol/mg protein, p≦0.05) and calcium citrate (761±59.4 vs 302.1±47.8 nmol/mg protein, p≦0.05).

### Effect of AC on PCNA Expression as Compared to Calcium Carbonate and Calcium Citrate

The PCNA is a 36 kDa molecular weight protein also known as cyclin. The protein has also been identified as the polymerase-associated protein and is synthesized in early G1 and S phases of the cell cycle. In early S phase, PCNA has a very granular distribution and is absent from the nucleoli. At late S phase, PCNA is prominent in the nucleoli. Hence, PCNA can be used as marker for DNA synthesis and cell proliferation.

The expression of PCNA in AC and calcium citrate treated cells was considerably higher than the control and calcium carbonate-treated cells. Addition of vitamin D₃ to the AC treated cells showed increased expression of PCNA compared to calcium carbonate+vitamin D₃ treated cells, suggesting the possible role of AC as an effective calcium supplement and its absorption is high in presence of vitamin D3.

Effect of AC on DNA Synthesis with the Comparison of Calcium Carbonate and Calcium Citrate

[³H] thymidine incorporation into DNA is the most commonly used method for assessing DNA synthesis and, thereby cellular proliferation. The counts were significantly more in AC treated cells when compared to the control (210.6±23.4vs51±14.7 cpm, p≦0.05), calcium carbonate (210.6±23.4vs62.8±11.6 cpm, p≦0.05) and calcium citrate (210.6±23.4vs45±9.9 cpm, p≦0.05), suggesting a possible role of AC in DNA synthesis and cell proliferation. Incubation of cells with both vitamin D₃ and AC resulted in more synthesis of DNA when compared to calcium carbonate+vitamin D₃ (388.9±19.6vs278.8±74.31 cpm, p≦0.05) and calcium citrate+vitamin D3 (388.9±19.6vs164.2±11.32 cpm, p≦0.05), indicating that AC can be absorbed more than other calcium sources in presence of vitamin _{D3}. This might indicate a superior bioavailability of AC over other the two calcium supplements.

### Effect of AC on Calcium Deposition or Mineralization as Compared to Calcium Carbonate and Calcium Citrate

After 2 days of treatment, calcium deposition, (i.e. mineralization) was found to be increased in AC-treated cells compared to the control (0.2580±0.016 vs 0.1190±0.0025 OD at 561 nm, p≦0.05), calcium carbonate (0.2580±0.016 vs 0.1810±0.0115 OD at 561 nm, p≦0.05) and calcium citrate (0.2580±0.016 vs 0.1267±0.0054 OD at 561 nm, p≦0.05) treated cells. Moreover, the mineralization capacity of AC was found to be higher in presence of vitamin D₃ as compared to calcium carbonate+vitamin D₃ (0.395±0.0216 vs 0.2037±0.0058 OD at 561 nm, p≦0.05) and calcium citrate+vitamin D₃ (0.395±0.0216 vs 0.1717±0.0071 OD at 561 nm, p≦0.05).

### Effect of AC on H₂O₂-Induced Oxidative Stress as Compared to Calcium Carbonate and Calcium Citrate

To determine the effect of AC on H₂O₂ induced oxidative damage to osteoblastic hFOB 1.19 cells, lipid peroxide (malondialdehyde; MDA) levels were assessed. 0.3 mM H₂O₂ treatment increased the MDA levels more than control osteoblasts (1.992±0.116 vs 0.6768±0.084 μM, p≦0.05). These results suggest that H₂O₂-enhanced ROS generation can damage lipids in osteoblast cells. However, levels of MDA in H₂O₂+AC-treated osteoblast cells were decreased compared to only H₂O₂-treated cells (1.992±0.116 vs 0.8295±0.009 μM, p≦0.05). Moreover, the anti-lipid peroxidative potential of AC was better than the other two calcium supplements, i.e., calcium carbonate (1.263±0.062 vs 0.8295±0.009 μM, p≦0.05) and calcium citrate (1.289±0.109 vs 0.8295±0.009 μM, p≦0.05 uM), respectively. These results indicate that AC can also reduce oxidative damage and stress in osteoblast cells.

This Example demonstrates that natural marine calcium supplement; AC can serve as a superior and bioavailable calcium supplement than the inorganic calcium sources. The effect of AC may be due to its content of other bone supporting minerals and their influence on ALP, PCNA and DNA synthesis; helping in the proliferation and mineralization of the osteoblast cells. Moreover, the anti-osteoporotic capacity of AC is more potent in presence of vitamin D₃. Furthermore, AC significantly reduces oxidative stress in osteoblast cells as demonstrated by reduced MDA levels.

### Example 13

A total of 100 women aged 29-80 (average 55.3 yrs) completed an informed consent, a baseline Dual-energy X-ray Absorptiometry test (DXA), Quality of Life Inventory (QOL), and a blood test panel of: hemoglobin A1C, glucose, insulin and circulating Vitamin D levels. No significant differences were found between the two study groups on any of the baseline measures. Upon completion of all baseline tests, subjects were randomly assigned to one of two groups:
1. a group receiving a calcium carbonate tablets containing 800 IU of Vitamin D₃ and 1,200 mg of calcium; and
2. a group receiving capsules containing 1,000 IU of Vitamin D₃ and 750 mg of calcium, magnesium, strontium, vanadium, and silica, derived from a rhodolith coralline algae.

Subjects consumed the supplements for 6 months and were paid a fee of $2.00/day for recording how many of the capsules or tablets they consumed each day, when they consumed them, and any adverse effects or discomfort they experienced. At the conclusion of the six-month study period, subjects completed the same tests taken at baseline and an anonymous questionnaire confirming the accuracy of the data they reported during the study.

Within the first three weeks of the study, 9 subjects in the carbonate group complained of adverse effects and asked to be removed from the study or changed to the plant-sourced supplement. None of the subjects taking the algae supplement asked to be removed from the study. A comparison in responses to a 9-item sub-scale on the QOL measuring gastro-intestinal revealed that, as compared to the average scores of algae subjects, subjects in the carbonate group reported more discomfort on all nine items and significantly more discomfort on two items (P=0.04 and P=0.02). These differences and loss of 18% of the subjects taking the carbonate supplement suggests taking the algae supplement resulted in lower discomfort ratings and increased tolerability.

Contrary to an expected annualized change in Bone Mineral Density (BMD) of ˜1%, there were no changes in baseline BMD in either group nor were there any statistically significant changes in BMD between the two study groups.

These data suggest that the algae supplement can achieve the same levels of efficacy as the carbonate supplement but with only 62.5% of the calcium and greater amounts of Vitamin D₃. Thus, the per-mg effect of the algae supplement was 1.6 times greater than the leading calcium supplement.

### Example 12

Subjects who had expressed an interest in participating in a bone health study were split into two groups and were given a total body dual-energy x-ray absorptiometry (DXA) test measuring bone mineral density (BMD), 43-test blood chemistry profile, and a quality of life (QOL) test. Subsequent to the testing, and without receiving test results, subjects reviewed the components of the bone health plan (Plan), and an informed consent that set forth the requirements of a 6-month open-labeled protocol.

In the first group (Group 1) of 274 potential subjects, 158 agreed to participate and signed the informed consent. A total of 125 completed the study per protocol (PP). Two weeks after the last subject in Group 1 completed the study, the same procedure was followed with a second group (Group 2) of 80 potential subjects, 58 of whom agreed to participate and 51 of whom completed the study PP.

Subjects were provided with an original (Algae-1) and a revised (Algae-2) version of an algae-based mineral complex. The Algae supplement is an algae-sourced form of calcium derived from a rhodolith coralline algae. The Plan also included strontium citrate tablets that were to be taken immediately before retiring (see Table):

| Ingredient or Component | Group 1 | Group 2 |
|---|---|---|
| Strontium Citrate (mg) | 680 | 680 |
| Total Algae Supplement (mg) | 2,400 | 2520 |
| Minerals in Supplement (mg) | 1,698 | 1,783 |
| Calcium (mg) | 720 | 756 |
| Magnesium (mg) | 72 | 350 |
| Vitamin D-3 (IUs of Cholecalciferol) | 800 | 1,600 |
| Vitamin K-2 as MK-4 (mg) | 1.5 | 0 |
| Vitamin K-2 as MK-7 (mcg) | 0 | 100 |
| Boron (mg) | 0 | 3 |
| Vitamin C (mg) | 0 | 50 |

To improve health literacy, subjects were provided with a booklet designed to improve health literacy that included calorie estimation charts and charts providing the glycemic load of over 300 common foods designed to increase the quality of carbohydrate intakes. In addition to the booklet, subjects were asked to wear a pedometer and to record their daily step totals at the end of each day to provide increased awareness of the physical activity levels.

Changes in BMD were measured using DXA at baseline and six months from baseline. To compare changes in mean BMD over different study periods, data were converted to MAPC. Actual changes in BMD were compared with expected changes using age-and-gender-adjusted data from studies of thousands of men and women that had established normal or expected annual changes for age and gender. These studies demonstrate that BMD increases with age until about the mid-thirties, remains constant for a few years and then progressively declines about 1% per year for women and about 0.5% for men.

To evaluate the safety of the Plan, the 43-chemistry blood test and a 50-item Quality of Life inventory were administered to all study participants at baseline and at the end of six months.

We measured compliance to the supplement usage using participants' daily tracking forms, a post-study anonymous questionnaire to report actual supplement usage, and a blinded subjective evaluation by the research technician(s) with whom the subject had the most frequent contact. The subjective evaluation rated compliance using a five point scale with 5 indicating near-perfect compliance and 1 reflecting poor compliance with the protocol. Participants with a 5 rating were classified as “compliant” and those with scores of 1, 2, 3 or 4 as “partially compliant.”

Continuously distributed data were summarized with the mean and standard deviation; binary outcomes were summarized with counts and percents. Groups (Group 1, Group 2) were contrasted on MAPC with analyses of covariance with adjustment for age and sex. Group contrasts with regard to binary outcomes were made with Pearson's chi-square. All statistical testing was 2-sided with a significance level of 5%. SAS Version 9.1.3 for Windows (SAS Institute, Cary, N.C.) was used throughout.

Both groups experienced a significant positive mean annualized percent change in BMD (MAPC) compared to expectation [Group 1: 1.15%, p=0.001; Group 2: 2.79%, p=0.001]. Both groups experienced a positive MAPC compared to baseline, but only Group 2 experienced a significant change [Group 1: 0.48%, p=0.14; Group 2: 2.18%, p<0.001]. The MAPC in Group 2 was significantly greater than that in Group 1 (p=0.005). The MAPC contrast between compliant and partially compliant subjects was significant in both groups (p=0.001 and p=0.003 respectively) with compliant subjects experiencing greater increases in MAPC than partially compliant. No clinically significant changes in blood chemistries or self-reported quality of life were found in either group. There were no significant baseline differences between the two groups in mean BMD or in variables related to BMD (age, sex, height, weight, percent fat, fat mass, or lean mass). Neither group showed significant baseline differences in BMD or related variables between volunteers and non-volunteers or between those who completed PP and those who were lost to attrition.

Following the Plan for six months with either version of the bone health supplement was associated with improvements in MPAC. Increased compliance was associated with greater increases as was modifying the bone health supplement with different amounts and types of nutrients, while holding all other components of the Plan constant. No adverse effects were reported in either group.

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

We claim:

1. A composition comprising:
a. at least 15%, by weight, of calcium derived from a rhodolith coralline algae;
b. magnesium;
c. strontium;
d. vanadium;
e. vitamin K; and
f. vitamin D3.

2. A composition according to claim 1 wherein the composition comprises at least 25%, by weight, calcium.

3. A composition according to claim 1 wherein the algae is non-geniculate.

4. A composition according to claim 1 wherein at least a portion of the magnesium, strontium, vanadium, and silica are derived from a rhodolith coralline algae.

5. A composition according to claim 1 wherein the vitamin K is vitamin K-2.

6. A composition according claim 1 wherein the composition comprises at least one mineral selected from iron, manganese, potassium, sulfur, zinc, and combinations thereof.

7. A method of treating osteoporosis, arthritis, bone spurs, plantar fasciitis, or other musculoskeletal disorders by ingesting a composition according to claim 1.

8. A kit comprising:
a. a composition according to claim 1; and
b. instructions for ingesting the composition.
